(19) United States
(12) Patent Application Publication
(10) Pub. No.: US 2022/0042977 A1
(43) Pub. Date: Feb. 10, 2022

(54) Protein Solubility Screening Kits and Their Use

(71) Applicant: ProStabilis, Inc., Cambridge, MA (US)

(72) Inventors: Ramil Latypov, Cambridge, MA (US); Yongwu Yang, Cambridge, MA (US)

(73) Assignee: ProStabilis, Inc., Cambridge, MA (US)

(21) Appl. No.: 16/984,863

(22) Filed: Aug. 4, 2020

(51) Int. Cl. G01N 33/537; G01N 33/84

(52) U.S. Cl. CPC G01N 33/5375; G01N 2400/18; G01N 33/84

(57) ABSTRACT

The present invention provides a multitude of biomacromolecule solubility screening kits, and methods of using such kits. Such kits provide a substantial improvement over presently available kits and methods and provide a substantial decrease in the amounts of biomacromolecules required to run such solubility screening.

### BACKGROUND

Therapeutic proteins and monoclonal antibodies continue to represent one of the fastest growing classes of biopharmaceutical products. Because of their involvement in different biochemical reactions, proteins naturally exhibit extreme variability in surface properties, stability, and solubility. Solution conditions that help stabilize (or solubilize) one protein do not necessarily apply to another protein. In practice, formulation of a given protein has to be adjusted on a case-by-case basis, which tends to be a lengthy and tedious process. The work of a formulation scientist consists of screening various solution conditions to improve protein stability and solubility. This area of applied research remains challenging, especially when a highly concentrated dosage form (>150 mg/mL) is desired. The higher a protein concentration in a sample, the more likely it is to exhibit an instability, such as aggregation, particulation, or phase separation. Screening of a sufficiently wide range of solution conditions is needed to develop a high quality formulation product. In a typical scenario, such screening effort may begin with preparation of 10-20 different highly concentrated protein samples for stability studies. This is often difficult to do because of the following: (1) highly concentrated proteins are very viscous and tend to precipitate on the ultrafiltration membrane, which leads to loss of material; (2) highly concentrated protein solutions exhibit solute specific Donnan effects making it difficult to attain the desired final concentration of excipients because of preferential binding or exclusion from a protein surface. An additional drawback is that this can be exceptionally consumptive (as it requires hundreds of grams of a highly purified material) and slow to a point of being inefficient. Traditionally overcome by brute force, these challenges ultimately limit the number of solution variables available for screening. As a result, typical development studies tend to fall short of providing sufficient coverage of the formulation space.

The lengthy and inefficient traditional formulation development can limit the number of assets that a company is able to move through its development pipeline. There are several established methods for assessing protein solubility briefly described herein. The most direct method is the ultrafiltration method that uses a semi-permeable membrane to upconcentrate protein sample above its solubility limit, followed by an estimation of protein concentration in the supernatant. This is one of the most consumptive and lengthy approaches, which is rarely used when the objective is to screen multiple formulation conditions (>>3). Another method is the second virial coefficient B₂₂ method (B₂ and A₂₂ can also be used)¹, which estimates the pair-wise interaction between protein molecules. Basically, the sign of B₂₂ serves as an indication of the presence of dominant repulsion or attraction between proteins in a given solution condition. A higher throughput but less rigorous modification of the B₂₂ method is an extrapolation method that uses dynamic light scattering and is sometimes called the interaction parameter or a coefficient of diffusion (k_{D}) method². In this method, a series of samples is prepared in the concentration range limited to 1-20 mg/mL to determine the dependence of the protein diffusion coefficient on protein concentration. The slope of the linear fit is then presented as the interaction parameter with stable and unstable conditions identified based on the sign and the magnitude of k_{D}. The disadvantage of these two methods is two-fold: 1) their scientific principle is based on the measurement of a pair-wise protein-protein interaction that may not accurately represent the collective inter-protein interaction in a concentrated protein solution; and 2) the B₂₂ and k_{D} measurements require relatively large amounts of protein and special sample preparation techniques including filtration and concentration. One more experimental approach to protein solubility includes measurement of protein concentration over its crystals³. Unfortunately, there is not a single universal solution that can result in crystallization of all proteins. Conversely, it cannot be expected that protein crystals would grow quickly or in sufficient amounts for solubility testing in all formulations. Protein solubility can also be assessed via precipitation with some salts, such as ammonium sulfate. The problem with this approach is the ionic nature of ammonium sulfate and the need to use it at high concentrations. Protein-protein interactions assessed with this method represent a case where a high conductivity environment suppresses long-range electrostatic interactions. This creates a problem with extrapolating the results of salt-based precipitation to low conductivity formulations typically used in pharmaceutical products.

An opportunity to accelerate development lies in a quick upfront identification of promising formulation conditions using smaller amounts of protein than are traditionally used, for example, by the methods set forth above. Once this is achieved, traditional formulation stability studies can be set up with greater confidence. Therefore, there is need for a more rapid, non-consumptive pre-screening tool predictive of protein solubility issues. When used prior to the initiation of a traditional stability study, such pre-screening can aid in establishing protocols for the stability studies and provide better leads.

Protein solubility is related to the inter-protein attraction, which is at the core of many types of instability exhibited by protein solutions. Suboptimal solubility in concentrated protein solutions is often the reason for precipitation, gelation, crystallization, and liquid-liquid phase separation (LLPS). Among these manifestations, LLPS stands out as a phenomenon that is well characterized, highly reproducible, and relatively fast. In contrast to real time aggregation or particle formation that may require long incubation periods (on the order of months to years), LLPS occurs and reaches equilibrium within minutes. LLPS is a spontaneous thermodynamic process that is sensitive to the formulation composition, sample temperature, and protein concentration. It usually occurs in highly concentrated formulations under conditions dominated by overall inter-protein attraction. Changes to formulation composition, such as pH adjustment or addition of excipients, may promote or mitigate LLPS by increasing or decreasing protein-protein attraction. A good example is formulating near the isoelectric point of a protein where attraction dominates repulsion leading to aggregation and precipitation. Because LLPS is related to the energy of inter-protein attraction and the latter determines protein solubility, it is critical to find a method able to distinguish the propensity of a given protein solution to undergo LLPS. The identification of such a method and its conversion into a fast, economic, and reproducible solubility screening tool is in the scope of the current invention.

LLPS allows to quickly navigate through the formulation space as it does not require lengthy incubations and it spontaneously proceeds towards equilibrium. LLPS in a given solution can be induced most readily by the addition of a crowding agent such as polyethylene glycol (PEG), which is a nonionic and chemically inert polymer that acts as a non-specific protein precipitant. Proteins have been shown to self-interact in the presence of PEG in a manner consistent with their behavior in the absence of PEG⁴. The role of PEG in the PEG-induced LLPS method (PEG-LLPS) is that of a universal modulator of the overall protein-protein attraction, and it has been successfully used to assess solubility of proteins and peptides in various solution conditions⁵⁻⁷. The greatest practical benefit of using PEG is that in its presence LLPS can be observed in very dilute samples (<1 mg/mL protein), which saves protein material. Without PEG, LLPS may sometimes be precluded by solution freezing even if preparation of a sufficiently concentrated sample (>100 mg/mL protein) was successful. All of the above make PEG-LLPS the method of choice to quickly and thoroughly explore the formulation space using little amount of protein.

Despite the fact that no other current approach outperforms PEG-LLPS with respect to the relevancy of data output, applicability across various proteins and formulations, and reduced sample consumption, the PEG-LLPS method is used on a regular basis only within a few academic labs and well-resourced biopharmaceutical companies. A more widespread use of it in formulation development is hampered by several factors that the current invention overcomes. Not all users are aware that PEG-LLPS experiments must closely follow a procedure that minimizes massive precipitation and sequestering of proteins in a non-equilibrium state. Users need to optimize PEG concentration for their measurements and avoid mixing highly concentrated PEG solutions with highly concentrated proteins. After mixing, a brief incubation period is recommended to re-solubilize any precipitated protein prior to starting the measurement. The subsequent cooling process must proceed with a ramp rate that provides sufficient time for equilibration. The sample dilution ratio must ensure no interference caused by buffer components and other solutes from the protein stock. Much attention needs to be paid to the formulation sample preparation, including appropriate PEG molecular weight selection, to ensure reproducible results. More importantly still, although the possible formulation space for this method is exceptionally large, there has been no convenient way of creating that many samples rapidly and reliably. This is the main reason the PEG-LLPS method is still under-utilized in formulation despite its outstanding potential. There is an unmet need to give users the ability to create a large enough number of formulation conditions for PEG-LLPS measurements. Once the formulation matrix is ready, the users would only need to provide their proteins of interest to perform these measurements with confidence and ease. This can be achieved by supplying specially configured pre-made reagent kits that help avoid tedious sample preparation, thereby enabling broad application of the method in protein solubility screening.

### REFERENCES

1. Neal B L, Asthagiri D, Lenhoff A M. (1998) Molecular origins of osmotic second virial coefficients of proteins. Biophys. J. 75. 2469-2477.
2. Saito S, Hasegawa J, Kobayashi N, et al. (2012) Behavior of monoclonal antibodies: relation between the second virial coefficient (B2) at low concentrations and aggregation propensity and viscosity at high concentrations. Pharm. Res. 292. 397-410.
3. Ries-Kautt M, Ducruix A. (1997) Inferences drawn from physicochemical studies of crystallogenesis and pre-crystalline state. Methods Enzymol. 276.23-59.
4. Wang Y, Lomakin A, Latypov R F, Laubach J P, Hideshima T, Richardson P G, Munshi N C, Anderson K C, Benedek G B. (2013) Phase transitions in human IgG solutions. J. Chem. Phys. 139(12). 121904. doi: 10.1063/1.4811345.
5. Wang Y, Lomakin A. Kanai S, Alex R. Benedek G B. (2017) Liquid-Liquid Phase Separation in Oligomeric Peptide Solutions. Langmuir: the ACS Journal of Surfaces and Colloids. 33, 7715-7721.
6. Thompson R W Jr, Latypov R F, Wang Y, Lomakin A. Meyer J A, Vunnum S, Benedek G B. (2016) Evaluation of effects of pH and ionic strength on colloidal stability of IgG solutions by PEG-induced liquid-liquid phase separation. J. Chem. Phys. 145, 185101.
7. Razinkov V I, Kleemann G R. (2016) High-throughput formulation development of biopharmaceuticals: practical guide to methods and applications. Woodhead Publishing.

### SUMMARY

The present invention offers specially designed reagent kits comprising different formulations with various concentrations of at least one crowding agent such as, for example and without limitation, polyethylene glycol (PEG) for conducting thermodynamically controlled protein precipitation. It provides reagent matrices that span an expanded formulation space and their method of use. This invention plays a foundational role for a new formulation development workstream of protein solubility optimization across many conditions via high-throughput PEG-LLPS. The discrete PEG zone design with each zone comprising a different PEG concentration makes the kits generally applicable for any protein molecule and potentially other types of biomacromolecules.

This kit invention provides a universal method for screening solubility of various protein classes, regardless of the size, amino acid sequence, or three-dimensional structure. It carries a transformative potential as it makes possible the same day identification of the best solution condition (as a function of pH, buffers, and any combinations thereof, with all major types of excipients) using dilute protein samples (0.5-5 mg/mL). This is expected to result in improved functioning and greater output of daily activities of organizations involved in formulation development, as well as greater quality and improved characteristics of the final therapeutic dosage forms.

Accordingly, one aspect of the present invention provides a kit for the screening of biomacromolecule solubility comprising:
deposited in the wells of a microplate, at least one range of core pH conditions, such range having sufficient pH variability to evaluate solubility of such biomacromolecules.

Also provided is a kit according to the previous aspect, further comprising:
at least one of the groups consisting of:
i. at least one crowding agent, having at least one concentration of such at least one crowding agent, wherein each such concentration of crowding agent being overlayed on the at least one range of core pH conditions;
ii. at least one crowding agent, having at least one concentration of such at least one crowding agent, wherein each such concentration of crowding agent being overlayed on at least one range of core pH conditions, and at least one tonicity modifying agent, wherein such tonicity modifying agent being overlayed on at least one range of core pH conditions and across each of the at least one concentration of such crowding agent;
iii. at least one crowding agent, having at least one concentration of such at least one crowding agent, wherein each such concentration of crowding agent being overlayed on at least one range of core pH conditions, and at least one additional different millimolar concentration of the same buffers being overlayed on at least one range of core pH conditions and across each of the at least one concentration of such crowding agent;
iv. at least one crowding agent, having at least one concentration of such at least one crowding agent, wherein each such concentration of crowding agent being overlayed on at least one range of core pH conditions, and at least one of the compounds selected from the group consisting of an amino acid, a sugar, and combinations thereof, each such selected amino acid, sugar, and combination thereof being overlayed on at least one range of core pH conditions and across each of the at least one concentration of such crowding agent; and
v. at least one crowding agent, having at least one concentration of such at least one crowding agent, wherein each such concentration of crowding agent being overlayed on at least one range of core pH conditions, and at least one of the compounds selected from the group consisting of a cyclodextrin and a surfactant, each such selected cyclodextrin and surfactant being overlayed on at least one range of core pH conditions and across each of the at least one concentration of such crowding agent.

Further provided is a kit for the screening of biomacromolecule solubility comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions.

An additional aspect provides for a kit for screening of biomacromolecule solubility comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one tonicity modifying agent wherein such tonicity modifying agent being overlayed on at least one range of core pH conditions and across each of the at least one concentration of such crowding agent.

Further provided is a kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one additional different millimolar concentration of the same buffers being overlayed on at least one range of core pH conditions and across each of the at least one concentration of such crowding agent.

A further aspect provides for a kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one of the compounds selected from the group consisting of an amino acid, a sugar, and combinations thereof, each such selected amino acid, sugar, and combination thereof being overlayed on at least one range of core pH conditions and across each of the at least one concentration of such crowding agent.

Also provided is a kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one of the compounds selected from the group consisting of a cyclodextrin and a surfactant, each such selected cyclodextrin and surfactant being overlayed on at least one range of core pH conditions and across each of the at least one concentration of such crowding agent.

Another aspect of the present invention provides for a kit for screening the solubility of biomacromolecules comprising:
aliquots of:
a. at least one range of core pH buffers; and
b. one or more selected from the groups consisting of:
   i. at least one crowding agent;
   ii. at least one tonicity agent;
   iii. at least one additional different millimolar concentration of the same buffers;
   iv. at least one amino acid;
   v. at least one cyclodextrin; and
   vi. at least one surfactant;
each in sufficient quantity to prepare at least one kit of the present invention.

The biomacromolecules frequently screened using the kits of the present invention are, without limitation, proteins; and the analytical method is frequently, without limitation, LLPS. References to protein or proteins herein are not limited to protein, per se, and, in fact, represent all biomacromolecules.

### BRIEF DESCRIPTION OF THE DRAWINGS

Features of the present invention will be more fully appreciated by reference to the following detailed description when taken in conjunction with the following drawings in which:

FIG. 1. depicts a flowchart of the Bottom Up protein solubility screening where formulation development proceeds through steps that directly inform each other to gain an insight into the protein behavior. The solid and dotted arrows serve to identify recommended and optional screening steps, respectively. The dashed arrows identify examples of the pre-made reagent kits required for each Tier.

FIG. 2a. depicts a sample layout of 4 PEG zones in a 96-well microplate for Tier 1 measurements (Kit Type 1).

FIG. 2b. depicts another sample representation of the Kit Type 1 layout for Tier 1, where b1 through b24 denote the same buffers as in Table 1.

FIG. 3. depicts a sample formulation layout for tonicity modifier screening in a 96-well microplate for Tier 2 measurements (Kit Type 2).

FIG. 4. depicts a sample formulation layout for buffer concentration optimization in a 96-well microplate for Tier 3 measurements (Kit Type 3).

FIG. 5. depicts a sample formulation layout for amino acids vs sugar (polyol, in this instance) screening in a 96-well microplate for Tier 4 measurements (Kit Type 4).

FIG. 6. depicts a sample formulation layout for cyclodextrin vs surfactant screening in a 96-well microplate for Tier 4 measurements (Kit Type 5).

FIG. 7a. depicts a Top Down approach to formulation development that prioritizes screening across the most effective solubilizing conditions presented on the Kit Types 4 and 5.

FIG. 7b. depicts Hybrid approaches that allow PEG zone mixing across several (up to 2 or more) reagent kits to explore conditions along any formulation variable or combinations thereof.

FIGS. 8a and b. depict sample layouts of 4 PEG zones in the case of the narrow pH Kit Types 1A and 1B designed for Tier 1 measurements.

FIGS. 9a and b. depict sample formulation layouts for tonicity modifier screening in the case of the narrow pH Kit Types 2A and 2B designed for Tier 2 measurements.

FIGS. 10a and b. depict sample formulation layouts for buffer concentration optimization in the case of the narrow pH Kit Types 3A and 3B designed for Tier 3 measurements.

FIGS. 11a and b. depict sample formulation layouts for amino acids vs sugar (polyol, in this instance) screening in the case of the narrow pH Kit Types 4A and 4B designed for Tier 4 measurements.

FIGS. 12a and b. depict sample formulation layouts for cyclodextrin vs surfactant screening in the case of the narrow pH Kit Types 5A and 5B designed for Tier 4 measurements.

While the aspects of the present disclosure are susceptible to various modifications and alternative forms, specific embodiments thereof are shown by way of example in the drawings and will herein be described in detail. It should be understood, however, that the drawings and detailed description are not intended to limit the disclosure to the particular forms illustrated but, on the contrary, the intention is to cover all modifications, equivalents and alternatives falling within the spirit and scope of the present disclosure as defined by the appended claims. The headings used herein are used for organizational purposes only and are not meant to limit the scope of the description. As used throughout this application, the word “may” is used in a permissive sense, meaning: “having the potential to”; rather than the mandatory sense meaning: “must”. Similarly, the words “include”, “including” and “includes” mean including, without limitation. Additionally, as used in this specification and the appended claims, the singular forms “a”, “an” and “the” include singular and plural referents unless the content clearly dictates otherwise.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combinations of features. In particular, with reference to the appended claims, features from dependent claims may be combined with those of independent claims and features from respective independent claims may be combined in any appropriate manner and not merely in the specific combinations enumerated in the appended claims.

### DETAILED DESCRIPTION

### Definitions

The term “biomacromolecules” means biomacromolecules, for example and without limitation, proteins, peptides, protein conjugates (such as antibody-drug conjugates and the like, regardless of whether covalently or non-covalently bound), nucleic acids, virus-like particles, viruses and the like.

The term “buffer” has its traditional meaning in the chemical arts and can include, for example and without limitation, any of the buffers set forth in Table 1, and the like.

The term “crowding agent” has its traditional meaning in the protein solubility art and can include, for example and without limitation, PEG (of various molecular weights, also well known in the art but can include, for example and without limitation, PEG 600, PEG 1000, PEG 8000 and the like), ammonium sulfate, linear or highly branched inert polymers or co-polymers, any one of a variety of dextran solutions, also known in the art, poloxamer 188 and other types of poloxamers of different size and composition, hydrophilic polysaccharides which dissolve readily in aqueous solution (e.g., Ficoll® products; Millipore Sigma, St. Louis, Mo., USA), and the like.

The term “cyclodextrin” has its traditional meaning in the pharmaceutical arts and can include, for example and without limitation, any member of a family of cyclic oligosaccharides and their derivatives (including, for example and without limitation, alpha-cyclodextrin, beta-cyclodextrin, gamma-cyclodextrin, sulfobutylether-beta-cyclodextrin (aka. Captisol® products; Ligand Pharmaceuticals Inc., San Diego, Calif., USA), 2-hydroxypropyl-beta-cyclodextrin, and the like).

The term “sugar” means: (1) carbohydrates (for example and without limitation, sucrose, trehalose, maltose, glucose (aka. dextrose), and the like); (2) polyalcohols (including, for example and without limitation, polyols and sugar alcohols, such as glycerol, mannitol, sorbitol, xylitol, and the like); and (3) polysaccharides (including, for example and without limitation, alginic acid and its different salts, hydroxypropyl methylcellulose (HPMC), various types of starch, and the like).

The term “surfactant” has its traditional meaning in the pharmaceutical arts and can include, for example and without limitation, any detergent or emulsifier, and combinations thereof (including, for example and without limitation, polysorbate-20 (aka. Tween-20), polysorbate-80 (aka. Tween-80), Triton X-100, poloxamers (e.g., Kolliphor® P 188 products; BASF, Florham Park, N.J., US), sodium dodecyl sulfate (SDS), and the like).

The terms “tonicity modifier” or “tonicity modifying agent” have their traditional meaning in the pharmaceutical arts and, in this instance include, for example and without limitation, at least one selected from the group of a salt and a sugar. More particularly, such agents include, for example and without limitation, inorganic and organic salts (such as sodium chloride, potassium chloride, sodium acetate, and the like).

### DESCRIPTION

The following description and examples are included to demonstrate the embodiments of the present disclosure. It should be appreciated by those of skill in the art that the compositions, techniques and methods disclosed in the examples herein function in the practice of the disclosed embodiments. However, those skilled in the respective arts should, in light of the present disclosure, appreciate that changes can be made to the specific embodiments and still obtain a like or similar result without departing from the spirit and scope of the disclosed embodiments.

The present specification includes references to “one aspect/embodiment” or “an aspect/embodiment”. These phrases do not necessarily refer to the same embodiment although embodiments that include any combination of the features or elements disclosed herein are generally contemplated unless expressly disclaimed herein. Particular compositions, features, processes, elements or characteristics may be combined in any suitable manner consistent with this disclosure.

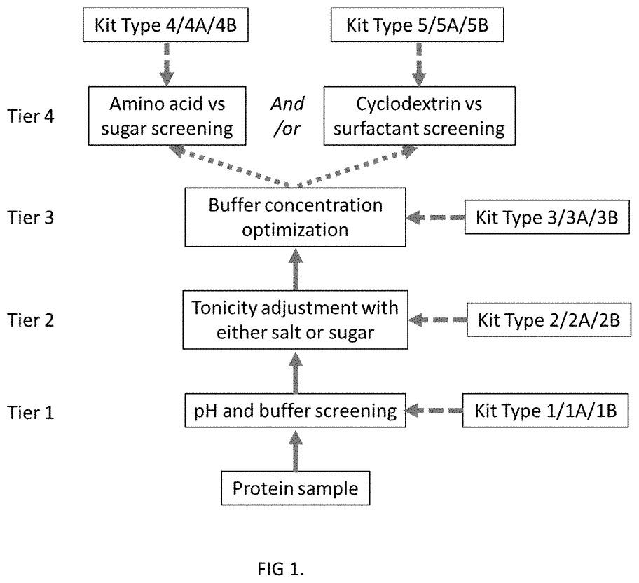

The traditional application of solubility screening via PEG, ammonium sulfate or other crowding agent precipitation suffers from a limited perspective on protein formulation development Available publications reflect disparate experimental approaches with no clear attempt to achieve alignment. Our unique approach to high-throughput formulation screening emphasizes PEG-induced LLPS (as a prescribed mode of protein precipitation), along with capturing the logic of formulation development. We offer an improvement to the prior art of formulation solubility screening in the form of a unified and regimented screening pathway. This pathway, as illustrated in FIG. 1, consists of four distinct stages or Tiers. The successful completion of each Tier is enabled by a particular PEG-LLPS reagent kit configuration of the current invention. This invention introduces the overarching concept as shown, step-by-step instructions with brief rationale, specific reagents and their exact placement on a 96-well plate as an example. This is a novel screening process that maximizes expeditious optimization of all major solution variables while minimizing protein consumption. There are a total of five different types of kit configurations in this invention denoted as Kit Type 1 through Kit Type 5 associated with specific Tiers (FIG. 1), and an additional kit that comprises aspects of each of the Kit Types 1 through 5 as optional elements of such additional kits. Other kits provide components to build the kits taught herein, and methods of using all such kits.

The foundational principle of the kit invention is a pre-defined set of core solution conditions encompassing what are defined as the most relevant pH and buffer conditions. This set is uniquely designed to maximize the useful data output with the smallest number of distinct conditions. By subjecting a protein to PEG-induced LLPS under this set of core conditions, and by following the workflow illustrated in FIG. 1, one can quickly optimize all major solution variables and thereby reduce the available solution space to a manageable size for subsequent studies.

Every Tier in FIG. 1 is supported by corresponding formulation kits as shown. The kits contain pre-made, ready-to-use formulation solutions dispensed into a sealed standard format microplate. Such seal is frequently aluminum foil, but other materials can be used. A user is required to transfer a pre-specified amount of solutions from one of the PEG zones (or combination thereof, as explained below) of the stock plate into an assay plate. Protein of interest is subsequently mixed in and the assay plate is gradually cooled to trigger PEG-LLPS. The plate is continuously monitored and upon completion of each experiment the temperature of protein phase separation is compared across all formulations and presented in the form of the protein solubility rank order.

The following is an example of 24 core formulations for wide pH screening. Other pH buffers and/or other excipients, other pH ranges and the number of core pH conditions each can be substituted for additional configurations of the present core pH-based conditions. Additionally, the kits set forth herein are not limited to 96-well microplates and can be used with microplates having any number of wells. As such, the examples set forth herein and the various kits set forth herein can be expanded to be included in larger microplates wherein multiple kit formats can be applied in one or more larger microplates.

Shown below is an example of 24 specific conditions in a 96-well microplate format. The 24 core conditions as presented serve to illustrate how one can encompass the pH range from about 4.5 to about 8.5, which is the most common pH range in protein formulation, by utilizing six commonly used buffers found in commercial formulations (acetate, succinate, citrate, histidine, phosphate, and tris(hydroxymethyl)aminomethane or tris). These core conditions are recommended to be put together in a particular way as to satisfy the following concepts:
1) the pH range is split into 0.5 pH unit increments to allow meaningful and systematic tracking of the effect of pH on protein solubility;
2) for every pH (with the exception of tris, pH 8.5), there exist two or more different buffers for direct comparison to aid in selection of a better buffering system;
3) the total number of different solution conditions is minimized to reduce protein consumption, without limiting the usefulness of data output;
4) the total number of solution conditions fits the 96-well plate configuration either vertically (as three columns) or horizontally (as two rows);
5) several replicas of the 24 core conditions can fit onto a 96-well plate in their entirety (without leaving empty wells or creating partial sets).

Examples of the 24 conditions for a wide pH screen are provided in Table 1. When these conditions are presented vertically (as three columns) or horizontally (as two rows), they form the building blocks of the following five wide pH reagent kit designs: Kit Type 1, Kit Type 2, Kit Type 3, Kit Type 4, and Kit Type 5. For brevity, only the vertical zone orientation is exemplified and discussed here. The same rationale is applicable to creating reagent kits with horizontally oriented zones. Within the scope of the current invention, the core conditions from Table 1 can be adjusted or modified to better suit the needs of solubility screening.

The high-throughput solubility screening based on PEG-LLPS requires adjustment of PEG concentration for a given protein. At least two PEG concentrations, low and high, is required to enable achievement of any intermediate PEG concentration via mixing. In all subsequent instances (and in all kit configurations), a PEG zone is comprised of the full set of 24 core conditions containing PEG at a certain (uniform) concentration, expressed as weight by weight percentage (% w/w).

TABLE 1
Examples of the 24 core conditions used
in the exemplified wide pH range kits

| Buffer | | | |
|---|---|---|---|
| ID | pH | Description | Wells in Kit Plate |
| b1 | 4.5 | 20 mM acetic acid-NaOH | A1, A4, A7, A10 |
| b2 | 5.0 | 20 mM acetic acid-NaOH | A2, A5, A8, A11 |
| b3 | 5.5 | 20 mM acetic acid-NaOH | A3, A6, A9, A12 |
| b4 | 4.5 | 20 mM succinic acid-NaOH | B1, B4, B7, B10 |
| b5 | 5.0 | 20 mM succinic acid-NaOH | B2, B5, B8, B11 |
| b6 | 5.5 | 20 mM succinic acid-NaOH | B3, B6, B9, B12 |
| b7 | 6.0 | 20 mM succinic acid-NaOH | C1, C4, C7, C10 |
| b8 | 4.5 | 20 mM citric acid-sodium citrate | C2, C5, C8, C11 |
| b9 | 5.0 | 20 mM citric acid-sodium citrate | C3, C6, C9, C12 |
| b10 | 5.5 | 20 mM citric acid-sodium citrate | D1, D4, D7, D10 |
| b11 | 6.0 | 20 mM citric acid-sodium citrate | D2, D5, D8, D11 |
| b12 | 6.5 | 20 mM citric acid-sodium citrate | D3, D6, D9, D12 |
| b13 | 5.5 | 20 mM histidine-HCl | E1, E4, E7, E10 |
| b14 | 6.0 | 20 mM histidine-HCl | E2, E5, E8, E11 |
| b15 | 6.5 | 20 mM histidine-HCl | E3, E6, E9, E12 |
| b16 | 7.0 | 20 mM histidine-HCl | F1, F4, F7, F10 |
| b17 | 6.0 | 20 mM sodium phosphate | F2, F5, F8, F11 |
| b18 | 6.5 | 20 mM sodium phosphate | F3, F6, F9, F12 |
| b19 | 7.0 | 20 mM sodium phosphate | G1, G4, G7, G10 |
| b20 | 7.5 | 20 mM sodium phosphate | G2, G5, G8, G11 |
| b21 | 8.0 | 20 mM sodium phosphate | G3, G6, G9, G12 |
| b22 | 7.5 | 20 mM tris-HCl | H1, H4, H7, H10 |
| b23 | 8.0 | 20 mM tris-HCl | H2, H5, H8, H11 |
| b24 | 8.5 | 20 mM tris-HCl | H3, H6, H9, H12 |

Kit Types 1 through 5 are exemplary of the type of kits disclosed herein. Each of these kits can be modified and combined pursuant to the teachings herein. Accordingly, broadly taught, a kit of the present invention includes a kit for the screening of biomacromolecule solubility comprising: deposited in the wells of a microplate, at least one range of core pH conditions, such range having sufficient pH variability to evaluate solubility of such biomacromolecules; and further comprising:
at least one of the group consisting of:
i. at least one crowding agent, having at least one concentration of such at least one crowding agent, wherein each such concentration of crowding agent being overlayed on the at least one range of core pH conditions;
ii. at least one crowding agent, having at least one concentration of such at least one crowding agent, wherein each such concentration of crowding agent being overlayed on at least one range of core pH conditions, and at least one tonicity modifying agent, wherein such tonicity modifying agent being overlayed on at least one range of core pH conditions and across each of the at least one concentration of such crowding agent;
iii. at least one crowding agent, having at least one concentration of such at least one crowding agent, wherein each such concentration of crowding agent being overlayed on at least one range of core pH conditions, and at least one additional different millimolar concentration of the same buffers being overlayed on at least one range of core pH conditions and across each of the at least one concentration of such crowding agent;
iv. at least one crowding agent, having at least one concentration of such at least one crowding agent, wherein each such concentration of crowding agent being overlayed on at least one range of core pH conditions, and at least one of the compounds selected from the group consisting of an amino acid, a sugar, and combinations thereof, each such selected amino acid, sugar, and combination thereof being overlayed on at least one range of core pH conditions and across each of the at least one concentration of such crowding agent; and
v. at least one crowding agent, having at least one concentration of such at least one crowding agent, wherein each such concentration of crowding agent being overlayed on at least one range of core pH conditions and at least one of the compounds selected from the group consisting of a cyclodextrin and a surfactant, each such selected cyclodextrin and surfactant being overlayed on at least one range of core pH conditions and across each of the at least one concentration of such crowding agent.

In this and other kits using a crowding agent, one concentration of such crowding agent can be used whereas a minimum of two concentrations of such crowding agent is typically more optimal.

### Kit Type 1: pH and Buffer Screening

Broadly, Kit Type 1 provides a kit for the screening of biomacromolecule solubility comprising:
a. deposited in the wells of a microplate, at least one range of core pH conditions, such range having sufficient pH variability to evaluate solubility of such biomacromolecules; and
b at least one crowding agent, having at least one concentration of such at least one crowding agent, wherein each such concentration of crowding agent being overlayed on at least one range of core pH conditions.

The utility and purpose of this kit is to enable wide pH and buffer optimization during Tier 1 screening (see FIG. 1).

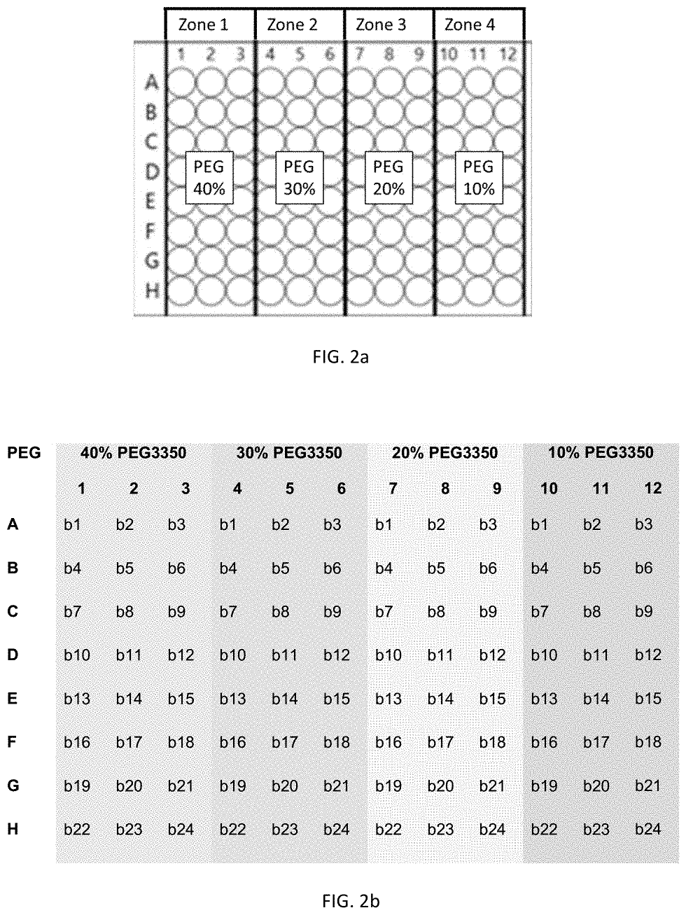

For example, a 96-well microplate can be divided into four different PEG (or other crowding agent) zones, as illustrated in FIGS. 2a and 2b for the design of the Kit Type 1. Each PEG zone contains the same 24 core conditions as in Table 1 with only the PEG (or other crowding agent) concentration changing. In the Kit Type 1, these zones contain, for example, PEG 3350 at 40, 30, 20 and 10%. 1:1 mixing by volume of the corresponding wells from either two of the PEG zones can create a new set of wells with an intermediate PEG concentration, which may be more optimal for a given protein. As an example, equal volume mixing of the 20% and 30% zones can create a new 25% PEG zone; unequal volume mixing allows creation of any other PEG concentration, if necessary. This mixing is conveniently performed using an 8-channel or a 12-channel pipettor or other means. Notably, it is necessary to maintain the order of the 24 conditions during mixing to ensure all other formulation characteristics (buffer identity and pH) remain the same.

The novelty of the Kit Type 1 concept lies in the fact that it defines the trajectory of all future formulation development because buffer and pH represent the most important parameters to be identified early. Taken together with its material sparing characteristics, it is an excellent fit for addressing the needs of candidate selection and early formulation development during Tier 1 solubility screening.

### Kit Type 2: Tonicity Modifier Screening

Broadly, Kit Type 2 provides a kit for screening of biomacromolecule solubility comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one tonicity modifying agent, wherein such tonicity modifying agent being overlayed on at least one range of core pH conditions and across each of the at least one concentration of such crowding agent.

The utility and purpose of this kit is to enable identification of an appropriate tonicity modifier during Tier 2 screening, as shown in FIG. 1.

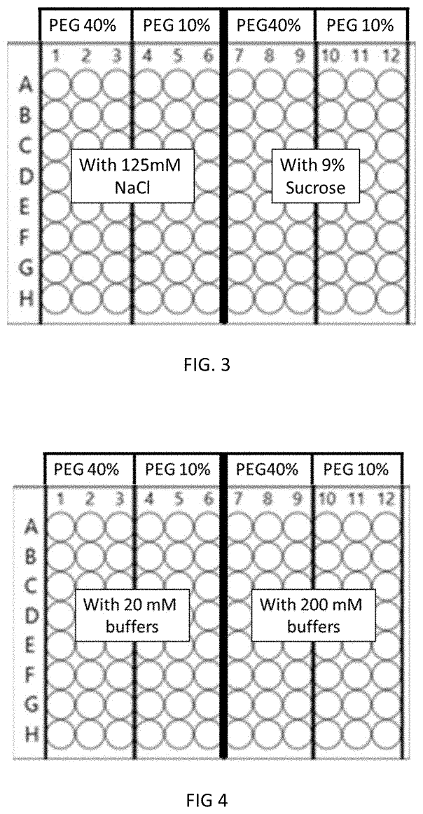

At least one crowding agent zone, typically at least two crowding agent zones, is minimally required to achieve screening conditions that accommodate any protein solubility. Kit Type 2 configuration is an adaptation of the Kit Type 1 to enable selection of a tonicity modifier. As one exemplification, this is accomplished by dividing, for example, the 96-well plate into two halves, each comprised of only two crowding agent zones, such as about 10% and about 40% PEG. These two zones contain the same, for example, 24 core conditions as in the Kit Type 1 (see Table 1). As illustrated in exemplary FIG. 3, one half of the plate is additionally supplemented with at least one salt including, for example, about 125 mM sodium chloride. The other half of the plate, for example, uses at least one sugar including, for example, about 9% w/v sucrose as a non-ionic tonicity modifier. Within each half, mixing of the two PEG zones can create any required crowding agent concentration for a given protein. As an example, mixing of equal volumes of 10% and 40% PEG can create a new 25% zone. Unequal volume mixing can create any other desired PEG zone, as necessary. All other characteristics of the 24 final solutions (buffer identity, buffer molarity, and pH) remain the same.

The novelty of the Kit Type 2 is in that it allows a side-by-side comparison of the salt- and sugar-adjusted isotonic formulations. The Kit Type 2 design is extremely flexible, as it allows assessing solution conditions where tonicity is simultaneously adjusted by salt and sugar. As an example, the equal volume mixing of a 125 mM sodium chloride matrix with a 9% sucrose matrix can create a new matrix of 62.5 mM sodium chloride with 4.5% sucrose. All other solution variables remain constant, such as the crowding agent percentage and the core buffers. A combination of tonicity modifiers may be of great practical interest. Sucrose that is usually needed for cryoprotection can raise the viscosity of protein solutions. This issue may be mitigated by the addition of sodium chloride, which was demonstrated to reduce viscosity. Taken together with its material sparing characteristics, Kit Type 2 is a good fit for identifying formulations for preclinical studies.

### Kit Type 3: Buffer Concentration Optimization

Broadly, Kit Type 3 provides a kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one additional different millimolar concentration of the same buffers being overlayed on at least one range of core pH conditions and across each of the at least one concentration of such crowding agent.

The utility and purpose of this kit is to enable buffer concentration optimization during Tier 3 screening.

Kit Type 3 is a modification of Kit Type 2, using, for example, the 24 core conditions, at least one crowding agent concentration of the same crowding agent with each overlayed on each of the at least one set of the 24 core conditions, and at least one additional concentration, typically, at least two concentrations, of the same buffers overlayed on each such concentration of the crowding agent.

For a more specific example of this Kit Type 3, each half of the plate has an overlay of one of the two buffer concentrations (that can be selected from the buffers shown in Table 1) on each of the two crowding agent concentrations. This example used solutions made of 20 mM buffers on one-half of the microplate, whereas the other half contains solutions made of 200 mM buffers (see FIG. 4). Similarly to Kit Type 2, it can use only two crowding agent zones within each half (10% and 40% in this example). This concept allows for a side-by-side comparison of the difference in protein solubility caused by an increase in buffer concentration. Most importantly, it is designed to assess solution conditions within the entire range of buffer concentrations from 20 mM to 200 mM. As an example, the equal volume mixing of the 20 mM buffer matrix with the 200 mM buffer matrix can create a new matrix of 110 mM buffers. With the obvious exception of buffer concentration, all other solution variables remain constant (crowding agent percentage, buffer identity, and pH). Assessing the effect of an adjustment in buffer concentration can be practically meaningful as changes in buffer concentration can produce changes in protein solubility. Some buffers, such as citrate, may be effective in modulating protein-protein interactions that govern aggregation and precipitation. Taken together with its material sparing characteristics, Kit Type 3 provides an option for a high-throughput refinement of formulation compositions during Tier 3.

Tier 4 represents a bifurcation point during typical formulation development. It is an advanced stage when formulators are equipped with information on the best buffer, pH, and tonicity modifier. At this stage, formulation developers are reasonably aware of the need to explore additional solution variables, such as, but not limited to, sugars (such as, for example and without limitation, sucrose, trehalose, glucose, sorbitol, mannitol, and the like), amino acids, cyclodextrins, and surfactants (including various detergents). The additional two kit configurations described below reflect what we believe represents the most efficient and informative use of PEG-LLPS during Tier 4.

### Kit Type 4: Amino Acids Vs Sugar Screening

Broadly, Kit Type 4 provides a kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, at least one of the compounds selected from the group consisting of an amino acid, a sugar, and combinations thereof, each such selected amino acid, sugar, and combinations thereof being overlayed on at least one range of core pH conditions and across each of the at least one concentration of such crowding agent.

The utility and purpose of this kit is to enable optimization along the amino acid—sugar axis during Tier 4 screening, as shown in FIG. 1.

For a more specific example of this Kit Type 4, Kit Type 4 is a modification of Kit Type 2 where one half of the plate contains the 24 core conditions supplemented with the 200 mM mixture of arginine and glutamic acid (where L-Arg and L-Glu are at the same molar concentration), whereas the other half is supplemented with 5% w/v sorbitol. Similar to Kit Type 2, it uses only two PEG zones (10% and 40%) within each half, as illustrated in FIG. 5. This concept allows for a side-by-side comparison of the difference in protein solubility caused by the use of these disparate reagents. Importantly, Kit Type 4 allows assessing solution conditions made up of the L-Arg/L-Glu and sorbitol mixed in any proportion. As an example, the equal volume mixing of the 200 mM L-Arg/L-Glu matrix with the 5% sorbitol matrix can create a new matrix of 100 mM L-Arg/L-Glu with 2.5% sorbitol. All other solution variables across all 24 core conditions remain constant (PEG percentage, buffer identity, and pH). Assessing the effect of these solution conditions on protein solubility can yield practically meaningful results. The need to perform such screening depends on the outcome of the Tier 1, 2 and 3 assessments. For example and without limitation, the L-Arg/L-Glu mixture (aka. Arginine Glutamate, or Glutargin) may be able to increase solubility of poorly soluble proteins. Sorbitol, on the other hand, can increase protein thermostability. Taken together with its material sparing characteristics, Kit Type 4 is a good fit for a high-throughput formulation optimization for a poorly behaving protein.

For the sake of clarity, other amino acids, or mixtures thereof, and other sugars can be used in place of those represented above.

### Kit Type 5: Cyclodextrin Vs Surfactant Screening

Broadly, Kit Type 5 provides a kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, at least one of the compounds selected from the group consisting of a cyclodextrin and a surfactant, each such selected cyclodextrin and surfactant being overlayed on at least one range of core pH conditions and across each of the at least one concentration of such crowding agent.

The utility and purpose of this kit is to enable optimization along the cyclodextrin—surfactant axis during Tier 4 screening, as shown in FIG. 1.

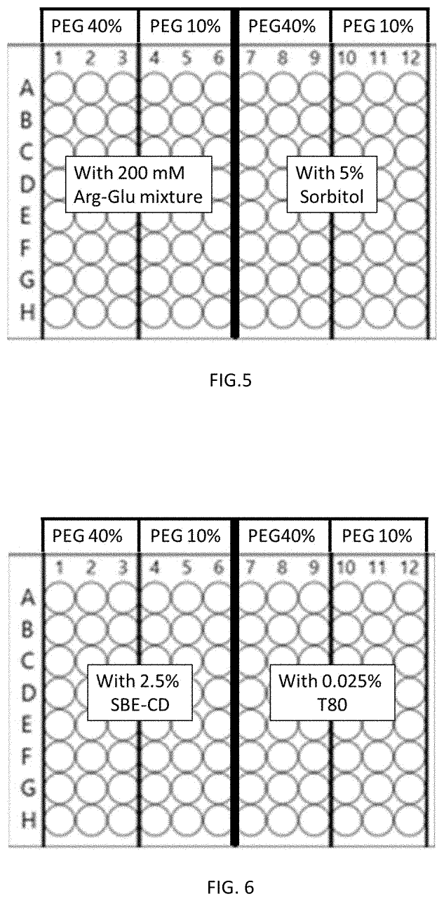

For a more specific example of this Kit Type 5, Kit Type 5 is a modification of Kit Type 2 where one half of the plate contains the 24 core conditions supplemented with 2.5% w/v sulfobutylether-beta-cyclodextrin (SBE-CD), and the other half is supplemented with 0.025% w/v Tween-80 (T80, also known as polysorbate-80). Similar to Kit Type 2, it can use only two PEG zones (e.g., 10% and 40%) within each half, as illustrated in FIG. 6. This concept allows for a side-by-side comparison of the difference in protein solubility caused by the use of these very different surface-active excipients. Importantly, Kit Type 5 allows assessing solution conditions made up of SBE-CD and T80 mixed in any proportion. As an example, the equal volume mixing of the 2.5% SBE-CD matrix with the 0.025% T80 matrix can create a new matrix of 1.25% SBE-CD with 0.0125% T80. All other solution variables across all 24 core conditions remain constant (PEG percentage, buffer identity, and pH). Assessing the effect of these solution conditions on protein solubility can be quite revealing. The need to perform such screening depends on the outcome of the Tier 1, 2 and 3 assessments. Taken together with its material sparing characteristics, Kit Type 5 is a good fit for a high-throughput formulation optimization for a protein prone to aggregate and/or form particles.

For the sake of clarity, other cyclodextrins and other surfactants can be used in place of those represented above. Furthermore, such surfactants can be selected from anionic, cationic, zwitterionic, and non-ionic surfactant classes.

Each of the kits disclosed herein can optionally include one or more microplates in which to carry out the intent of such kits.

Additional kits provided herein include aliquots of the various elements used in the kits disclosed herein. Accordingly, the present invention also provides a kit for screening the solubility of biomacromolecules comprising:
aliquots of:
a. at least one range of core pH buffers; and
b. one or more selected from the groups consisting of:
   i. at least one crowding agent;
   ii. at least one tonicity agent;
   iii. at least one additional different millimolar concentration of the core pH buffers;
   iv. at least one amino acid;
   v. at least one cyclodextrin; and
   vi. at least one surfactant;

each in sufficient quantity to prepare at least one kit of the present invention. The various elements can comprise one or more of the compounds disclosed in each class of elements discussed herein, or equivalents thereof, and can be used at the concentrations exemplified or otherwise, at the discretion of the end user.

The case-by-case nature of protein formulation development necessitates that the screening process remains highly adaptive. The way adaptability is achieved within the scope of the current invention is further illustrated here.

The Bottom Up approach shown in FIG. 1 is the most rational screening process as it sequentially proceeds through steps that directly inform each other. In this figure, the solid and dashed arrows serve to identify recommended and optional screening steps, respectively. It makes sense to begin with the wide pH screen in the absence of other excipients (as in Tier 1) to understand the pH dependence of protein solubility. Subsequent iterative screening each time a new excipient is added serves to verify that the pH dependence of protein solubility has not changed. This approach is recommended for formulation scientists working with relatively standard molecules that behave in a reasonably predictive way. Unexpected findings regarding solubility can be made even within a well-known class of proteins as a result of amino acid sequence variation, which may require a case-by-case formulation optimization. The advantage of the highly regimented step-by-step Bottom Up process is that it is able to yield a more complete and thorough understanding of the protein behavior.

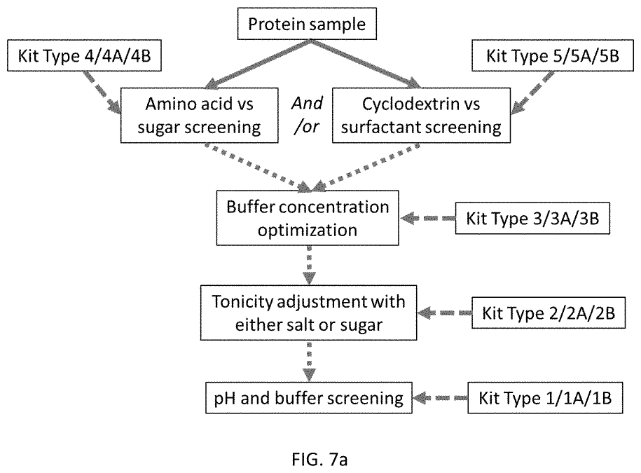

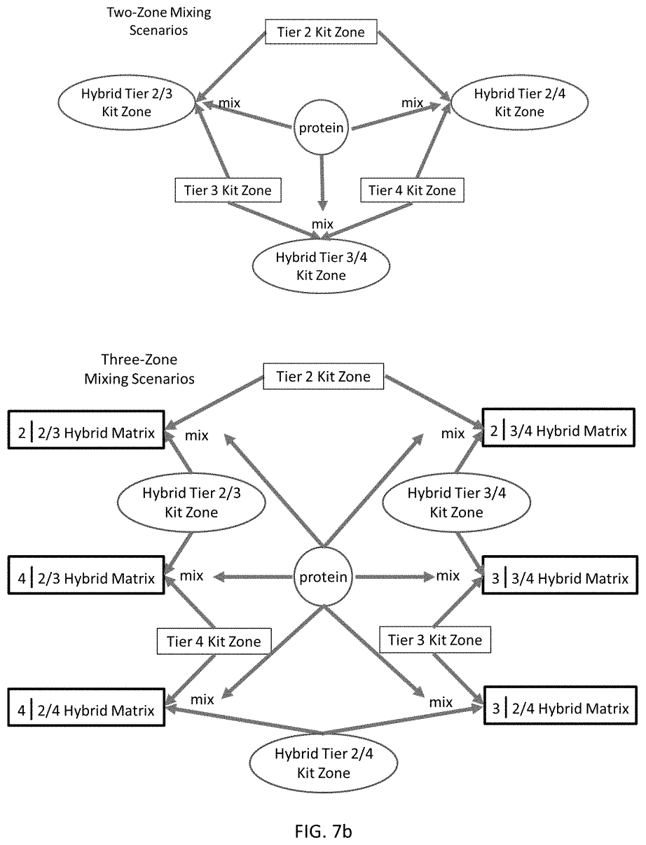

The systematic Bottom Up approach may not always be necessary, especially when the alternative Top Down (see FIG. 7a) and Hybrid (see FIG. 7b) approaches prove to be more efficient. In the case of the Top Down and the Hybrid approaches, a protein that is anticipated to behave poorly is immediately screened against some of the most effective solubilizing conditions presented on the Kit Types 4 and 5. Once these conditions are tested and shown to be effective at raising solubility, the purpose of any additional PEG-LLPS-based screening would be of confirmatory nature (as shown by the optional dashed arrows). The Top Down approach could be applied to a protein in the absence of prior knowledge of an effective remedy for raising solubility. In such a case, the outcome of the very first screening step may be sufficient to inform the direction of future formulation development.

The Hybrid approach applies when formulators are aware of how to improve solubility, yet the available pre-made kits do not offer the required excipient combination. The current invention utilizes common sets of 24 solution conditions across the different reagent kits, with each set representing a separate PEG zone. The 24 solution conditions can be increased or decreased in number if desired and when kits are not pre-prepared and only the reagents are provided in a kit or reagents are blended from other prepared kits. A given crowding agent zone can be mixed with another same crowding agent zone either from the same reagent kit or from another reagent kit or even from several (2 or more) other reagent kits. The goal of mixing crowding agent zones across different reagent kits is to explore formulation conditions along any formulation variable(s), or combination thereof, regardless of whether they are provided by the same reagent kit. Because of this built-in flexibility, the Hybrid approach allows creation of any new matrix of solubility screening conditions. Within the scope of the current invention, it represents the most adaptive approach to probe an extremely wide formulation space. As an example, the sugar matrix of the Kit Type 4 can be mixed with the surfactant matrix of the Kit Type 5 to create a brand new matrix containing both sugar and surfactant. A combination of sugar and surfactant is in fact a popular approach to formulating monoclonal antibodies. As another illustration, a tonicity modifying agent matrix of the Kit Type 2 can be mixed simultaneously with the buffer concentration and the cyclodextrin matrices of the Kits Type 3 and Type 5, respectively. The result of this mixing would be an absolutely unique set of 24 formulation conditions that may simultaneously provide cryoprotection, raise solubility, and reduce particulation. The uniqueness of the reagent configurations of the current invention circumvents the limitations of conventional screening methods while significantly reducing protein material need or sample preparation time.

The following is an example of formulation sets for narrow pH screening.

Each of the five aforementioned main kit designs (Kit Type 1 through Kit Type 5) can be modified to give rise to two respective narrow pH kit configurations. These progenic configurations are denoted with letters A or B to identify acidic and basic pH ranges. As an example, Kit Type 1 related narrow pH configurations are Kit Type 1A and Kit Type 1B, with all of these kits (Kit Type 1, Kit Type 1A, and Kit Type 113) fulfilling the objectives of Tier 1 screening (FIG. 1).

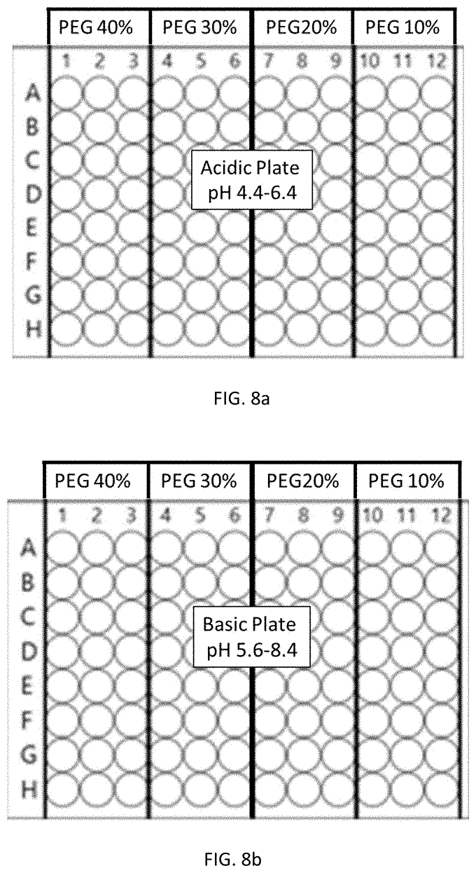

The rationale behind the narrow pH kit configurations is to enable a more precise identification of optimal pH for future development. This is achieved by reducing the pH interval from 0.5 to 0.25 units. The original pH range of 4.5 to 8.5 is split into the following ranges: acidic (pH range from 4.4 to 6.4) and basic (pH range from 5.6 to 8.4), as shown in FIGS. 8a, and 8b.

In the narrow pH kits, for example and without limitation, the six buffers (acetate, succinate, citrate, histidine, phosphate, and tris(hydroxymethyl)aminomethane or tris) are split into two distinct sets each comprised of three buffers. This allows appropriate coverage of the acidic and basic pH ranges while maintaining the same number of 24 core conditions per PEG zone. As an example, the way the narrow pH core conditions are put together can follow these principles:
1) the pH effect is assessed with a 0.25 pH unit increment to allow more precise identification of optimal pH, provided, however, the user of a kit of the present invention can adjust the pH unit increment to any increment that would be meaningful to such user for a given analysis (including, for example and without limitation, pH unit increments of 0.05, 0.10, 0.20.0.25, 0.30, 0.40, 0.50, 0.60 and the like, and any additional fraction thereof);
2) with the exception of acetate at pH 4.4, citrate at pH 6.2-6.4, histidine at pH 5.6-5.8, and tris at pH 8.2-8.4, all other pH conditions allow direct comparison of different buffering systems;
3) the total number of different solution conditions is minimized to reduce protein consumption, without limiting the usefulness of data output;
4) the total number of solution conditions fits the 96-well plate configuration either vertically (as three columns) or horizontally (as two rows);
5) several replicas of the 24 core conditions can fit onto a 96-well plate in their entirety (without leaving empty wells or creating partial sets).

Examples of chemical composition of the acidic and basic core conditions are provided in Tables 2a, and 2b. Whether presented vertically (as three columns) or horizontally (as two rows), these conditions form the building blocks of ten new reagent kits: Kit Type 1A, Kit Type 1B, Kit Type 2A, Kit Type 2B, Kit Type 3A, Kit Type 3B, Kit Type 4A, Kit Type 4B, Kit Type 5A, and Kit Type 5B. For brevity, only the vertical zone orientation is exemplified and discussed herein. The same rationale is applicable to creating reagent kits with horizontally oriented crowding agent (e.g., PEG) zones. Within the scope of current invention, these narrow pH conditions are not fixed and can be modified to better suit the needs of solubility screening for a given protein.

TABLE 2a
The composition of the 24 core formulations
for the acidic pH range.

| Buffer | | | |
|---|---|---|---|
| ID | pH | Description | Wells in Kit Plate |
| b1 | 4.4 | 20 mM acetic acid-NaOH | A1, A4, A7, A10 |
| b2 | 4.6 | 20 mM acetic acid-NaOH | A2, A5, A8, A11 |
| b3 | 4.8 | 20 mM acetic acid-NaOH | A3, A6, A9, A12 |
| b4 | 5.0 | 20 mM acetic acid-NaOH | B1, B4, B7, B10 |
| b5 | 5.2 | 20 mM acetic acid-NaOH | B2, B5, B8, B11 |
| b6 | 5.4 | 20 mM acetic acid-NaOH | B3, B6, B9, B12 |
| b7 | 4.6 | 20 mM succinic acid-NaOH | C1, C4, C7, C10 |
| b8 | 4.8 | 20 mM succinic acid-NaOH | C2, C5, C8, C11 |
| b9 | 5.0 | 20 mM succinic acid-NaOH | C3, C6, C9, C12 |
| b10 | 5.2 | 20 mM succinic acid-NaOH | D1, D4, D7, D10 |
| b11 | 5.4 | 20 mM succinic acid-NaOH | D2, D5, D8, D11 |
| b12 | 5.6 | 20 mM succinic acid-NaOH | D3, D6, D9, D12 |
| b13 | 5.8 | 20 mM succinic acid-NaOH | E1, E4, E7, E10 |
| b14 | 6.0 | 20 mM succinic acid-NaOH | E2, E5, E8, E11 |
| b15 | 4.6 | 20 mM citric acid-sodium citrate | E3, E6, E9, E12 |
| b16 | 4.8 | 20 mM citric acid-sodium citrate | F1, F4, F7, F10 |
| b17 | 5.0 | 20 mM citric acid-sodium citrate | F2, F5, F8, F11 |
| b18 | 5.2 | 20 mM citric acid-sodium citrate | F3, F6, F9, F12 |
| b19 | 5.4 | 20 mM citric acid-sodium citrate | G1, G4, G7, G10 |
| b20 | 5.6 | 20 mM citric acid-sodium citrate | G2, G5, G8, G11 |
| b21 | 5.8 | 20 mM citric acid-sodium citrate | G3, G6, G9, G12 |
| b22 | 6.0 | 20 mM citric acid-sodium citrate | H1, H4, H7, H10 |
| b23 | 6.2 | 20 mM citric acid-sodium citrate | H2, H5, H8, H11 |
| b24 | 6.4 | 20 mM citric acid-sodium citrate | H3, H6, H9, H12 |

TABLE 2b
The composition of the 24 core formulations
for the basic pH range.

| Buffer | | | |
|---|---|---|---|
| ID | pH | Description | Wells in Kit Plate |
| b1 | 5.6 | 20 mM histidine-HCl | A1, A4, A7, A10 |
| b2 | 5.8 | 20 mM histidine-HCl | A2, A5, A8, A11 |
| b3 | 6.0 | 20 mM histidine-HCl | A3, A6, A9, A12 |
| b4 | 6.2 | 20 mM histidine-HCl | B1, B4, B7, B10 |
| b5 | 6.4 | 20 mM histidine-HCl | B2, B5, B8, B11 |
| b6 | 6.6 | 20 mM histidine-HCl | B3, B6, B9, B12 |
| b7 | 6.8 | 20 mM histidine-HCl | C1, C4, C7, C10 |
| b8 | 7.0 | 20 mM histidine-HCl | C2, C5, C8, C11 |
| b9 | 6.0 | 20 mM sodium phosphate | C3, C6, C9, C12 |
| b10 | 6.2 | 20 mM sodium phosphate | D1, D4, D7, D10 |
| b11 | 6.4 | 20 mM sodium phosphate | D2, D5, D8, D11 |
| b12 | 6.6 | 20 mM sodium phosphate | D3, D6, D9, D12 |
| b13 | 6.8 | 20 mM sodium phosphate | E1, E4, E7, E10 |
| b14 | 7.0 | 20 mM sodium phosphate | E2, E5, E8, E11 |
| b15 | 7.2 | 20 mM sodium phosphate | E3, E6, E9, E12 |
| b16 | 7.4 | 20 mM sodium phosphate | F1, F4, F7, F10 |
| b17 | 7.6 | 20 mM sodium phosphate | F2, F5, F8, F11 |
| b18 | 7.8 | 20 mM sodium phosphate | F3, F6, F9, F12 |
| b19 | 8.0 | 20 mM sodium phosphate | G1, G4, G7, G10 |
| b20 | 7.6 | 20 mM tris-HCl | G2, G5, G8, G11 |
| b21 | 7.8 | 20 mM tris-HCl | G3, G6, G9, G12 |
| b22 | 8.0 | 20 mM tris-HCl | H1, H4, H7, H10 |
| b23 | 8.2 | 20 mM tris-HCl | H2, H5, H8, H11 |
| b24 | 8.4 | 20 mM tris-HCl | H3, H6, H9, H12 |

In each narrow pH kit configuration, a crowding agent zone is typically comprised of the full set of 24 core conditions (either acidic or basic) containing such crowding agent at a certain % w/w concentration. The following narrative briefly introduces various exemplifications of narrow pH kits along with their intended use. For more information refer to the corresponding wide pH kit sections.

For the sake of exemplification, the reagents and configurations thereof represented in these and other examples throughout this disclosure are not meant to limit the use of other reagents and/or configurations thereof.

### Kit Types 1A and 113: Narrow pH and Buffer Screening

The utility and purpose of these kits is to support narrow pH and buffer optimization during Tier 1 screening, as shown in FIG. 1.

In this case, for example and without limitation, a single 96-well plate holds four different crowding agent (e.g., PEG) zones, as illustrated in FIGS. 8a, 8b. The zones 1, 2, 3 and 4 correspond to the same 24 core conditions with only, in this example, the PEG 3350 concentration changing from 40 to 10%. Mixing of the corresponding wells from either two of such PEG zones can create a new set of wells (a new zone) with any intermediate PEG concentration, which may be more optimal for a given protein.

The Kit Types 1A and 1B can be used to validate findings from the Kit Type 1 wide pH measurement regarding the most suitable buffer and pH. Taken together with their material sparing characteristics, these kits are able to support lead candidate selection and early formulation development.

### Kit Types 2A and 2B: Tonicity Modifier Screening

The utility and purpose of these kits is to support identification of the tonicity modifier during Tier 2 screening, as shown in FIG. 1.

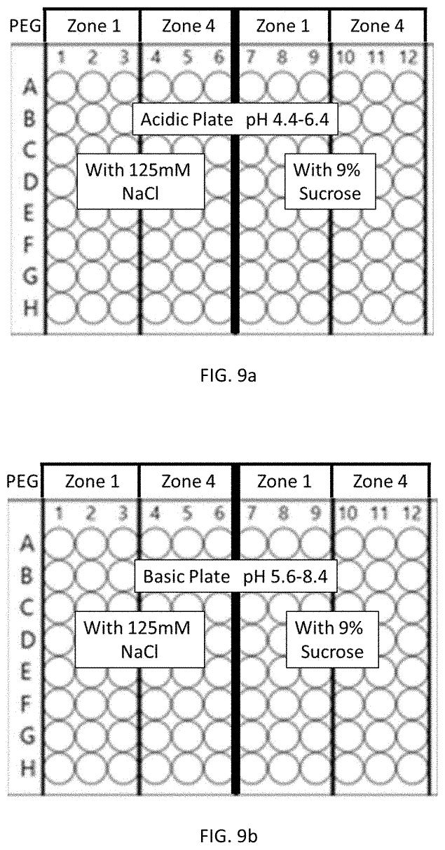

Kit Types 2A and 2B configurations are the derivatives of the Kit Types 1A and 1B designs modified to enable selection of a tonicity modifier. This is accomplished by dividing the 96-well plate format into two halves, each comprised of only two PEG zones, such as, for example, 10% and 40% PEG. These two zones contain the same 24 core conditions as in the Kit Types 1A and 1B, respectively. As illustrated in FIGS. 9a, 9b, one half of the plate is additionally supplemented with 125 mM sodium chloride, which represents the most commonly used ionic tonicity modifier. The other half of the same plate uses 9% w/v sucrose as a sugar-based non-ionic tonicity modifier.

Similar to the wide pH Kit Type 2, the narrow pH Kits 2A and 2B allow a side-by-side comparison of the salt- and sugar-adjusted isotonic formulations. These kits are just as adaptive to enable assessment of solution conditions where tonicity is simultaneously adjusted by both salt and sugar mixed in any proportion. They can be used to validate findings from the Kit Type 2 wide pH measurement regarding the most optimal tonicity modifier. Taken together with their material sparing characteristics, Kits 2A and 2B are good fits for identifying formulations for preclinical studies.

### Kit Types 3A and 3B: Buffer Concentration Optimization

The utility and purpose of these kits is to support buffer concentration optimization during Tier 3 screening, as shown in FIG. 1.

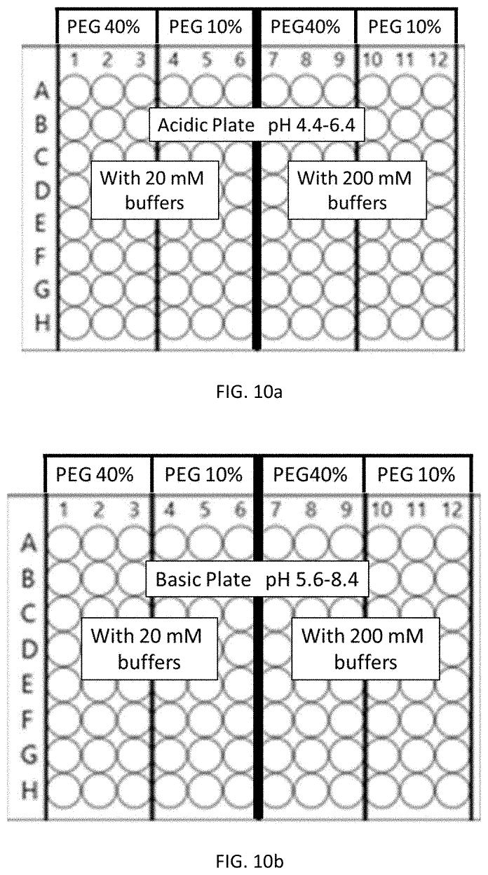

Kit Types 3A and 3B are modifications of the Kit Types 2A and 2B where one half of the plate contains the 24 core conditions made of 20 mM buffers, whereas the other half contains solutions made of 200 mM buffers (see FIGS. 10a, 10b). Similarly to the Kit Types 2A and 2B, their key feature is the use of, for example, only two PEG zones (i.e. with 10% and 40% PEG) in each half. This allows a side-by-side comparison of the difference in protein solubility caused by an increase in buffer concentration. Kit Types 3A and 3B are designed to assess solution conditions within the entire range of buffer concentrations from less than 20 mM to greater than 200 mM, typically between about 20 mM to about 200 mM. They can be used to validate findings from the Kit Type 3 wide pH measurement regarding the most optimal buffer concentration. Taken together with their material sparing characteristics, Kit Types 3A and 3B are good fits for a high-throughput refinement of formulation compositions during Tier 3 screening.

### Kit Types 4A and 4B: Amino Acids Vs Sugar Screening

The utility and purpose of these kits is to support optimization along the amino acid—sugar axis during Tier 4 screening, as shown in FIG. 1.

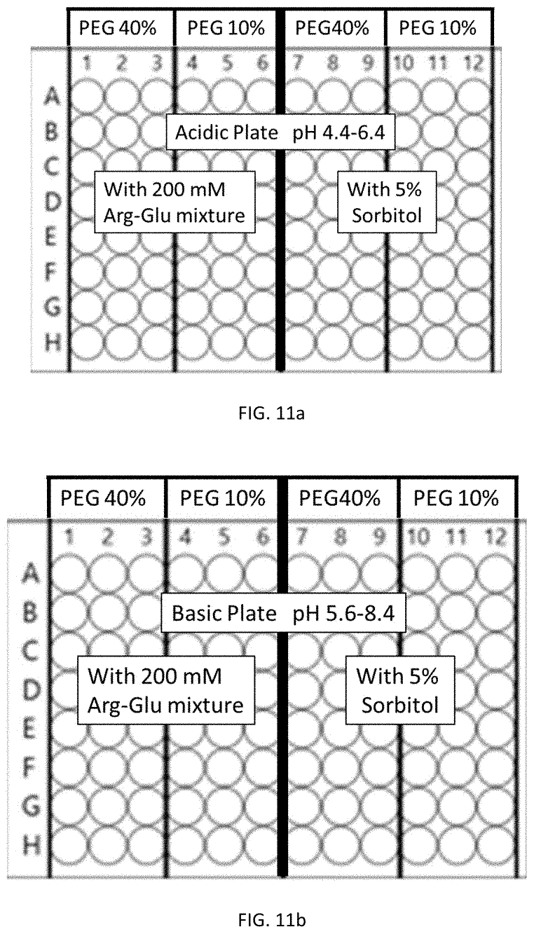

Kit Types 4A and 4B are modifications of the Kit Types 2A and 2B where, for example, one-half of the plate contains the 24 core conditions supplemented with the 200 mM mixture of arginine and glutamic acid (where L-Arg and L-Glu are at the same molar concentration), whereas the other half is supplemented with 5% w/v sorbitol. Similarly to the Kit Types 2A and 2B, their key feature is the use of only two PEG zones (i.e., with 10% and 40% PEG) in each half, as illustrated in FIGS. 11a, 11b. This design allows a side-by-side comparison of the difference in protein solubility caused by the use of these disparate reagents. Kit Types 4A and 4B can support assessing solution conditions made up of the L-Arg/L-Glu and sorbitol matrices mixed in any proportion. They can be used to validate findings from the Kit Type 4 wide pH measurement regarding the most optimal additional excipient(s), and combinations thereof. Taken together with their material sparing characteristics, Kit Types 4A and 4B are good fits for a high-throughput formulation refinement for a poorly behaving protein. For the sake of clarity, other amino acids, or mixtures thereof, and other sugars can be used in place of those represented above.

### Kit Types 5A and 5B: Cyclodextrin Vs Surfactant Screening

The utility and purpose of these kits is to support optimization along the cyclodextrin—surfactant axis during Tier 4 screening, as shown in FIG. 1.

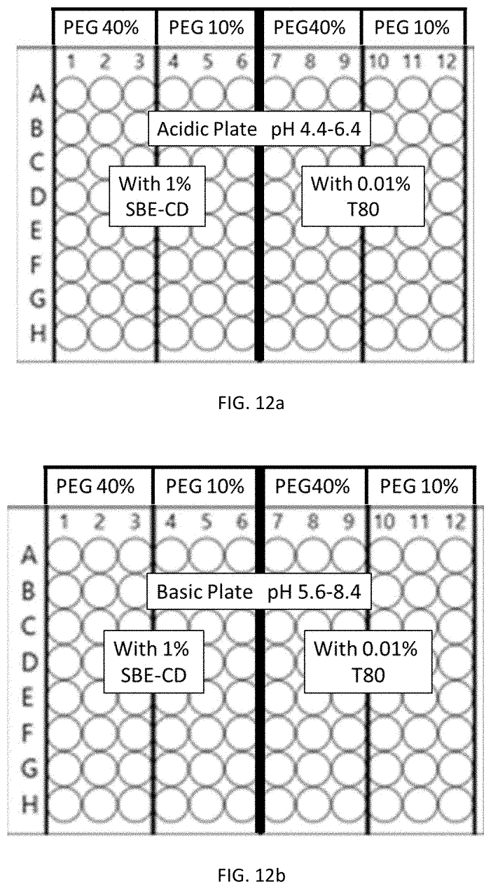

Kit Types 5A and 5B are modifications of the Kit Types 2A and 2B where one-half of the plate contains the 24 core conditions supplemented with 2.5% w/v sulfobutylether-beta-cyclodextrin (SBE-CD), whereas the other half is supplemented with 0.025% w/v Tween-80 (T80, aka. polysorbate-80), as illustrated in FIGS. 12a, 12b. Similar to the Kit Types 2A and 2B, their key feature is the use of only two PEG zones (i.e. with 10% and 40% PEG) in each half. This design allows for a side-by-side comparison of the difference in protein solubility caused by the use of these very different surface-active excipients. The Kit Types 5A and 5B can support assessing solution conditions made up of SBE-CD and T80 matrices mixed in any proportion. They can be used to validate findings from the Kit Type 5 wide pH measurement regarding the most optimal additional excipient(s), and combinations thereof. Taken together with their material sparing characteristics, Kit Types 5A and 5B are good fits for a high-throughput formulation refinement for a biomacromolecule prone to aggregate or form particles. For the sake of clarity, other cyclodextrins and other surfactants can be used in place of those represented above. Furthermore, such surfactants can be selected from anionic, cationic, zwitterionic, and non-ionic surfactant classes.

Kit reagents, microplate and design variables included in the present invention:
1) While the proposed kit format is a 96-well microplate [specifically, a standard deep-well (with 2 mL per well capacity) “mother” plate that harbors the reagents, and a 96-well assay “daughter” plate (0.1 mL per well; Greiner Bio-One 675096) to which reagents from the “mother” plate are transferred for experiment and to which protein is added for measurement], included in this disclosure are all possible “mother” plate configurations, such as number of wells per plate, fill volumes, and materials of construction, regardless of whether the “mother” plate is designed for multi- or single-use. In addition, the present disclosure includes all other possible means of reagent distribution, such as being supplied in separate reservoirs (bottles, tubes, vials, some forms of prefilled microarrays/microfluidics, etc.) or presented in different physical states (liquid, frozen liquid, lyophilizate or other dry powder, nanoparticles, microparticles, tablets, etc.). The idea behind marketing the pre-made reagents in a 2 mL stock “mother” plate is to allow users to perform multiple experiments with a single kit purchase (we estimate that 2 mL+0.1 mL will provide for up to 20 different experiments per one crowding agent zone (e.g., PEG zone)). The Greiner assay plate is an added consumable that may be optionally included in a kit of the present invention.
2) We elected to have four different PEG zones per 96-well plate in the case of the wide pH Kit Type 1, but other options exist such as having only one, two, or three PEG zones, as well as more than four within the same, for example, 96-well plate.
3) The chosen plate format (as examples, 48-well, 96-well, or 384-well plates) and the number of different crowding agent zones per plate (as in the previous point) determine the maximal number of possible solution conditions per one crowding agent zone (i.e. 96÷4=24). The variations of plates may be used to provide, for example, any number of solutions per zone (less than 24, as well as greater than 24).
4) The key to liquid-liquid phase separation induced by a crowding agent, particularly PEG (as in the PEG-LLPS method), is in the use of PEG of an appropriate molecular weight. There is a substantial number of commercially available PEG products that would satisfy this requirement. Essentially, all PEG formulations ranging in size from about 2 kDa to about 10 kDa would work. Although 3 kDa PEG (exact molecular weight: 3350 Da) is exemplified herein, PEG products that are smaller and larger than this size, and for any mixture of lower and higher molecular weight PEGs, may be used in the presently disclosed kits.
5) Other polymers similar to PEG, can also induce LLPS including, for example, PEG-like molecules and/or PEG derivatives (such as linear or branched, with additional chemical groups imparting some advantageous properties, and the like). Other crowding agents are set forth in the definitions above.
6) Ultimately, the choice of the polymer/crowding agent would dictate the selection of the exact polymer/crowding agent concentration within the zones. In the present wide pH Kit Type 1, PEG 3350 is used at the following weight by weight % concentrations: low PEG zone (10%), medium PEG zone (20%), high PEG zone (30%), and very high PEG zone (40%). This is a wide enough range that allows solubility assessment of proteins with very different intrinsic solubility, being universally applicable to any protein modality (from poorly soluble antibody fragments that may require very little PEG to cause LLPS to highly soluble full-length antibodies that require greater concentrations of PEG). For PEG 3350, the entire concentration range includes, for example, 1-50 w/w %. The same would apply to the 4 kDa PEG, as it is very close in size to PEG 3350. For a smaller molecular weight PEG, it would potentially require expanding the range to 1-60 w/w %. PEG 10 kDa, as an example of a much larger PEG polymer weight, cannot be dissolved at 50-60% without creating an excessively viscous liquid that is difficult to handle. Accordingly, the w/w percentage of PEG/crowding agent concentration is not made to be limiting.
7) The 24 solution conditions, which constitute a single PEG zone in the current wide pH kit design, cover a pH range from 4.5-8.5 that is the most commonly used range in formulation development. However, there could be cases where pH 3 to 4, or above 9 would be practically useful, or even a pH range from less than about 2 to pH greater than about 12 using any buffer that could achieve the desired target pH ranges, in any pH increment that would be limited only by the number of wells in a microplate and the kit variations included therein, consistent with configurations of the kits taught herein. In addition to single buffers, different buffers or components thereof could be mixed in different proportions to create single, dual, triple or (poly)-buffering systems with wider or unique buffering capacities. In fact, a number of universal (poly)-buffers exist that are not proprietary and would be able to cover the same pH range as in our kit. These may contain various amino acids (or other zwitterionic molecules with dual function in addition to pH buffering) on top of the traditional weak acids and bases. Accordingly, the present kits are not limited to the pH values, the pH increments and/or the buffers/buffer composition used to attain specific pH values as disclosed herein.
8) Similarly, other elements (e.g., pH, buffers, crowding agents, amino acids, cyclodextrins and the like), concentrations, ranges and/or configurations thereof as disclosed for use in the kits of the present invention may be readily substituted by equivalent elements, concentrations and/or combinations thereof and/or configurations used therewith. Accordingly, the elements, concentrations, ranges, and/or configuration thereof disclosed herein are not intended to be limiting to the present disclosure. Moreover, it is the elements, concentrations, ranges and configurations thereof, and their equivalents and variations that represent a novel approach to protein solubility screening using the kits and methods disclosed herein. Additionally, elements included in the present kits, or equivalents thereof, can be established in a “mother” microplate or other welled plate. An infinite number of concentrations of such elements may be used in assay plates via combinations of two or more concentrations of one or more such elements. Further, a user of the kits of the present invention can also produce an infinite number of combinations of such elements using well known techniques including, for example and without limitation, pipetting and combining at least two such elements of the infinite number of concentrations. Using the kits of the present invention in an iterative manner reduces the amount of protein sample required for analysis compared to traditional and presently known protein solubility analysis methods while providing a previously unprecedented amount of relevant information in a single experiment or series of experiments using the kits of the present invention.
9) Additionally, the kits of the present invention are not limited to analysis via LLPS. For example, there may be other possible precipitation reactions that may not be strictly defined as LLPS and, as such, may also present practical interest (i.e. related to development of therapeutic dosage forms based on amorphous precipitation, crystallization, gelation, nanoparticles, microparticles, etc.). In fact, it may sometimes be difficult to know or assess whether LLPS has truly occurred during solubility measurements.
10) While the primary use of the kits of the present invention is to select conditions that improve protein solubility, there are additional uses where the intent is to seek conditions that accelerate protein precipitation (e.g., in a search for low solubility conditions) if, for example, the goal is to develop a highly precipitated dosage form such as, for example, an amorphous suspension. Another example is the use of such kits with the goal of identifying conditions that selectively precipitate out a specific protein regardless of the solution matrix or whether some other proteins (or other macromolecules) are present, and whether such other macromolecules do or do not co-precipitate.
11) Starting protein concentration in the stock (that is to be provided by user) is recommended to be between 10-100 mg/mL. Target final (experimental) protein concentration is recommended to be 0.5-5 mg/mL as a result of a 20-fold dilution of the 10-100 mg/mL stocks (5 microLiters of a protein is mixed with 95 microLiters of the pH/buffer solution=100 microLiters per well in the assay plate). However, any protein concentration that would provide results using the kits of the present invention is acceptable.
12) Although the primary use of the kits of the present invention is for the analysis of proteins and antibodies, the present kits can also be used for analysis of any biomacromolecule (including, for example and without limitation, proteins, peptides, protein conjugates (such as antibody-drug conjugates, etc. regardless of whether covalently or non-covalently bound), nucleic acids, virus-like particles, and viruses).

We claim:

1. A kit for the screening of biomacromolecule solubility comprising:
deposited in the wells of a microplate, at least one range of core pH conditions, such range having sufficient pH variability to evaluate solubility of such biomacromolecules.

2. A kit according to claim 1, wherein the at least one range of core pH conditions is established in pH increments of at least 0.25 pH units.

3. A kit according to claim 1, wherein the at least one range of core pH conditions is established on a contiguous horizontal or vertical block of wells.

4. A kit according to claim 3, further comprising:
at least one of the groups consisting of:
i. at least one crowding agent, having at least one concentration of such at least one crowding agent, wherein each such concentration of crowding agent being overlayed on the at least one range of core pH conditions;
ii. at least one crowding agent, having at least one concentration of such at least one crowding agent, wherein each such concentration of crowding agent being overlayed on at least one range of core pH conditions, and at least one tonicity modifying agent, wherein such tonicity modifying agent being overlayed on at least one range of core pH conditions and across each of the at least one concentration of such crowding agent;
iii. at least one crowding agent, having at least one concentration of such at least one crowding agent, wherein each such concentration of crowding agent being overlayed on at least one range of core pH conditions, and at least one additional different millimolar concentration of the same buffers being overlayed on at least one range of core pH conditions and across each of the at least one concentration of such crowding agent;
iv. at least one crowding agent, having at least one concentration of such at least one crowding agent, wherein each such concentration of crowding agent being overlayed on at least one range of core pH conditions, and at least one of the compounds selected from the group consisting of an amino acid and a sugar, and combinations thereof, each such selected amino acid, sugar, and combination thereof being overlayed on at least one range of core pH conditions and across each of the at least one concentration of such crowding agent; and
v. at least one crowding agent, having at least one concentration of such at least one crowding agent, wherein each such concentration of crowding agent being overlayed on at least one range of core pH conditions, and at least one of the compounds selected from the group consisting of a cyclodextrin and a surfactant, each such selected cyclodextrin and surfactant being overlayed on at least one range of core pH conditions and across each of the at least one concentration of such crowding agent.

5. A kit according to claim 4 wherein said biomacromolecule is at least one protein.

6. A kit for the screening of biomacromolecule solubility comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions.

7. A kit according to claim 6, wherein said biomacromolecule is at least one protein.

8. A kit for screening of biomacromolecule solubility comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one tonicity modifying agent, wherein such tonicity modifying agent being overlayed on at least one range of core pH conditions and across each of the at least one concentration of such crowding agent.

9. A kit according to claim 8, wherein said biomacromolecule is at least one protein.

10. A kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one additional different millimolar concentration of the same buffers being overlayed on at least one range of core pH conditions and across each of the at least one concentration of such crowding agent.

11. A kit according to claim 10, wherein said biomacromolecule is at least one protein.

12. A kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one of the compounds selected from the group consisting of an amino acid, a sugar, and combinations thereof, each such selected amino acid, sugar, and combination thereof being overlayed on at least one range of core pH conditions and across each of the at least one concentration of such crowding agent.

13. A kit according to claim 12, wherein said biomacromolecule is at least one protein.

14. A kit for screening the solubility of biomacromolecules comprising:
a. deposited in the wells of a microplate, at least one range of core pH conditions, such range having sufficient pH variability to evaluate solubility of such biomacromolecules; and
b. at least one crowding agent, having at least one concentration of such at least one crowding agent, wherein each such concentration of crowding agent being overlayed on at least one range of core pH conditions, and at least one of the compounds selected from the group consisting of a cyclodextrin and a surfactant, each such selected cyclodextrin and surfactant being overlayed on at least one range of core pH conditions and across the at least one concentration of such crowding agent.

15. A kit according to claim 14, wherein said biomacromolecule is at least one protein.

16. A kit for screening the solubility of biomacromolecules comprising:
aliquots of:
a. at least one range of core pH buffers; and
b. one or more selected from the groups consisting of:
i. at least one crowding agent;
ii. at least one tonicity agent;
iii. at least one additional different millimolar concentration of the core pH buffers;
iv. at least one amino acid;
v. at least one cyclodextrin; and
vi. at least one surfactant;
each in sufficient quantity to prepare at least one kit of claim 4.

17. A kit according to claim 16, wherein:
a. said at least one range of core pH buffers comprises buffers selected from the group consisting of acetic acid-NaOH, succinic acid-NaOH, citric acid-sodium citrate, histidine-HCl, sodium phosphate, and tris-HCl;
b. said at least one crowding agent is PEG;
c. said tonicity agent selected from the group consisting of a sodium chloride as a salt and sucrose as a sugar;
d. said at least one amino acid is a mixture of arginine and glutamic acid;
e. said at least one sugar is selected from the group consisting of sucrose and sorbitol;
f. said cyclodextrin is sulfobutylether-beta-cyclodextrin; and
g. said at least one surfactant is polysorbate-80.

18. A method of reducing the amount of biomacromolecule required to conduct protein solubility screening compared to traditional methods of biomacromolecule screening comprising using a kit of claim 1.
